(19) United States
(12) Patent Application Publication
(10) Pub. No.: US 2008/0114314 A1
(43) Pub. Date: May 15, 2008

(54) CONNECTOR HANDLE FOR TUBING

(75) Inventors: Dirk Muehlhoff, Jena (DE); Dirk Preuss, Jena (DE)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: CARL ZEISS MEDITEC AG, Jena (DE)

(21) Appl. No.: 11/932,120

(22) Filed: Oct. 31, 2007

(30) Foreign Application Priority Data
Nov. 10, 2006 (DE) 102006053582.0

(51) Int. Cl. A61M 35/00

(52) U.S. Cl. 604/300

(57) ABSTRACT

The invention concerns a manual connector for a hose line to connect to a pressure or vacuum-generating device, where it is equipped with an additional functional element such as a filter, a fluid separator or an identification element.

### RELATED APPLICATION

The present application claims the benefit of priority to German Patent Application No. 10 2006 53 582.0 filed on Nov. 10, 2006. Said application is incorporated by reference herein.

### FIELD OF THE INVENTION

The invention concerns a manual connector for a hose line that is used to connect the hose line to a counterpart.

### BACKGROUND OF THE INVENTION

Such a hose line can be used in particular to transport fluid or gaseous media in medical technology, especially also as low pressure or vacuum pipe to suction off body fluids or surgical debris.

### SUMMARY OF THE INVENTION

The invention is particularly well suited to realize the suctioning of debris from the surgical field occurring during a laser cornea treatment or to maintain a vacuum during such a procedure. The lasers used here are especially femtosecond lasers.

During such an operation, the eye is affixed with a contact glass using vacuum, as described e.g. in U.S. Pat. No. 6,254,595 or U.S. Pat. No. 6,676,652. Since debris occurring during the laser treatment, such as lachrymal fluid, is suctioned off by vacuum and thereby could contaminate the device used to generate the vacuum, special measures are necessary to prevent the contamination of sterile parts of the treatment devices.

One option, as utilized by the patent holder of the above patents, Intralase Corp., Irvine, Calif., USA, with its device Intralase® FS, is to merely generate the vacuum mechanically, using a simple vacuum suction pump with pistons, which is connected to the contact glass by using a hose that is discarded together with the contact glass following the procedure.

If the vacuum, or the low pressure, is to be generated using a motor-driven suction pump, it must by all means be prevented that debris or fluids get into the suction pump; in this context, it is known to place a filter before the suction pump. Due to the required sterile environment, this is subject to special procedures, or it must be exchanged regularly, which requires considerable effort.

The invention addresses the problem of how to overcome the disadvantages described above and to provide a simple manually operated hose connector.

The problem is solved by the invention by providing a manual connector for a hose line to connect to a pressure- or vacuum-generating device with an additional functional element.

It represents an even greater advantage, if this additional functional element is a filter or a separator for water or liquid.

With a different aspect of the invention, this additional functional element can also be e.g. an RFID (Radio Frequency Identification) chip or a visually identifiable mark (e.g. color marking or similar).

The plug according to this invention is preferably fashioned as a locking Luer plug.

It represents an advantage, if the hose line cannot be disconnected from the manual connector.

Thereby, the invention assures a connection between the pressure or vacuum hose and the device used to generate the pressure or vacuum that is easy to handle and prevents that these become dirty or contaminated by the debris particles or fluids.

### BRIEF DESCRIPTION OF THE DRAWINGS

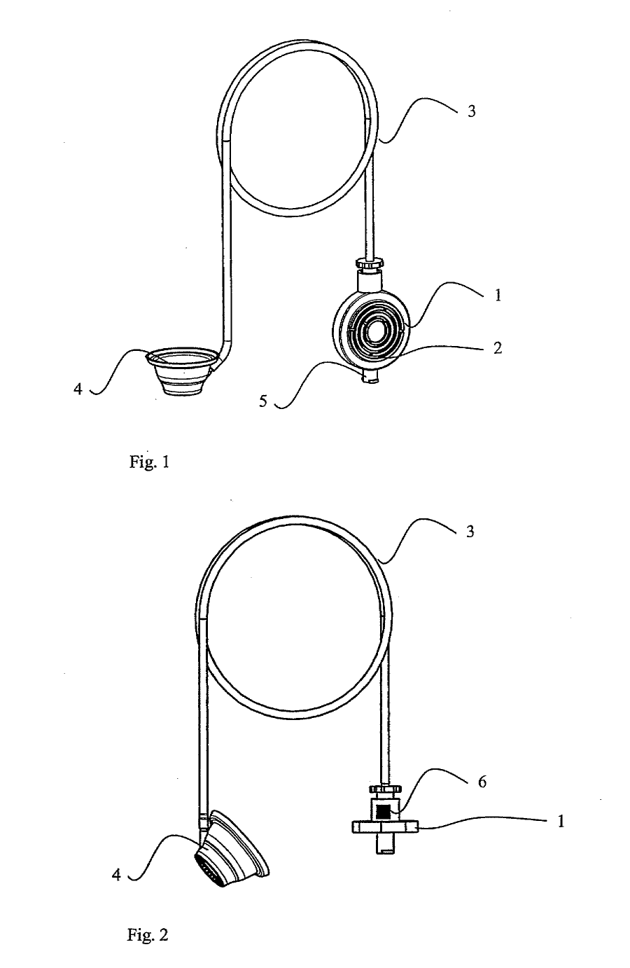

The invention is described below in two embodiments (FIGS. 1 and 2).

### DETAILED DESCRIPTION

FIG. 1 shows a manual connector (1) that contains a filter (2). This manual connector (1) is connected by a hose line (3) to a contact glass (4), as described in the printed publications U.S. Pat. No. 6,254,595 or U.S. Pat. No. 6,676,652, the content of which is hereby referenced. The plug is affixed with its distal connector 5 to a corresponding counterpart that is connected to the device generating the vacuum (not depicted here).

FIG. 2 shows a different embodiment of the manual connector (1), which is again connected to the contact glass (4) with a hose line (3). This manual connector is equipped with a RFID chip (6), which for example information about the characteristics of the contact glass may have been downloaded. In addition, this plug can have a built-in filter as well. This makes it possible to actualize a treatment set of simple design and cost-effective production, which can be easily connected and disconnected and which can also assure the cleanliness and sterility of the treatment device and the vacuum generator, as these treatment sets are customarily designed for one-time use.

The invention is not limited to the embodiments with the connection to a contact glass, as described, but encompasses in general the integration of an additional functional element in a manual connector for a hose line.

1. A treatment set for use in corneal treatment procedures in which a contact lens is applied to a cornea, in which the contact lens in fluid communication with a pressure or vacuum generating device and in which it is desired to maintain the contact lens in a sterile, uncontaminated state, the treatment set comprising:
the contact lens;
a fluid conduit connected in operable fluid communication with the contact lens; and
a manual connector in operable fluid communication with the fluid conduit, the manual connector comprising an integral functional element that prevents the transmission of contaminants between the contact lens and the pressure or vacuum generating device.

2. The treatment set according to claim 1, wherein the integral functional element comprises a filter.

3. The treatment set according to claim 1, wherein the integral functional element comprises a fluid separator.

4. The treatment set according to claim 1, further comprising an identification element.

5. The treatment set according to claim 2, further comprising an identification element

6. The treatment set according to claim 2, further comprising an identification element.

7. The treatment set according to claim 1, further comprising a locking Luer plug.

8. A manual connector to couple a fluid conduit in fluid communication between a pressure or vacuum generating device and an instrument in a clean and sterile field, the manual connector comprising:
an integral functional element that prevents the transmission of contaminants between the fluid conduit and the pressure or vacuum generating device.

9. The manual connector according to claim 8, wherein the integral functional element comprises a filter.

10. The manual connector according to claim 8, wherein the integral functional element comprises fluid separator.

11. The manual connector according to claim 8, further comprising an identification element.

12. The manual connector according to claim 8, further comprising a locking Luer plug.

13. A treatment set for use in corneal treatment procedures in which a contact lens is applied to a cornea, in which the contact lens in fluid communication with a pressure or vacuum generating device and in which it is desired to maintain the contact lens in a sterile, uncontaminated state, the treatment set comprising:
the contact lens;
a fluid conduit connected in operable fluid communication with the contact lens; and
a manual connector in operable fluid communication with the fluid conduit, the manual connector comprising an additional integral functional element.

14. The treatment set according to claim 13, wherein the integral functional element comprises an identification element.

15. The treatment set according to claim 13, wherein the integral functional element comprises a filter.

16. The treatment set according to claim 13, wherein the integral functional element comprises a fluid separator.
